(19) United States
(12) Patent Application Publication
(10) Pub. No.: US 2016/0303116 A1
(43) Pub. Date: Oct. 20, 2016

(54) TREATMENT OF MULTIPLE SCLEROSIS WITH A 1,2,4-TRIAZOLO [1,5A] PYRIDINE DERIVATIVE

(71) Applicant: CEPHALON, INC., Frazer, PA (US)

(72) Inventors: Pawel T. DOBRZANSKI, Dowingtown, PA (US); Joel KAYE, Netanya (IL); Arie ORBACH, Rehovot (IL); Bruce A. RUGGERI, West Chester, PA (US); Matthew M. SEAVEY, Secane, PA (US)

(21) Appl. No.: 15/102,896

(22) PCT Filed: Dec. 10, 2014
(86) PCT No.: PCT/US14/69495

§ 371 Date: Jun. 9, 2016

Related U.S. Application Data
(60) Provisional application No. 61/914,634, filed on Dec. 11, 2013.

(51) Int. Cl. A61K 31/496; A61K 9/00

(52) U.S. Cl. CPC A61K 31/496; A61K 9/0053

(57) ABSTRACT

The present disclosure provides methods and compositions for treating chronic autoimmune diseases, such as multiple sclerosis, using [8-(4-methanesulfonyl-phenyl)-[1,2,4]triazolo[1,5-a]pyridin-2-yl]-[3-(4-methyl-piperazin-1-yl)-phenyl]-amine or a pharmaceutically acceptable salt thereof.

### CROSS-REFERENCE TO RELATED APPLICATIONS

This application claims the benefit of and priority to U.S. Application No. 61/914,634, filed Dec. 11, 2013, the contents of which are incorporated by this reference, as if fully set forth herein.

### FIELD

The disclosure is directed to the therapeutic treatment of diseases via the administration of compounds and pharmaceutical compositions thereof.

### BACKGROUND

Multiple sclerosis (MS) is a chronic human autoimmune disease caused by an inflammatory reaction to myelin antigens in the central nervous system leading to demyelination and neurodegeneration. A substantial percentage of MS patients develop clinical paralysis and there is no curative therapy available.

Thus, there is a need for the development of safe and effective treatments for the debilitating conditions of multiple sclerosis. The disclosure is directed to these and other important needs.

### SUMMARY

The present disclosure provides methods for treating multiple sclerosis (MS) comprising identifying an individual having MS or an individual susceptible to the development of MS and administering Compound A to said individual

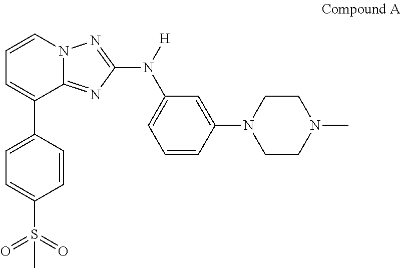 [SMILES: CN1CCN(c2cccc(Nc3nc4c(-c5ccc(S(C)(=O)=O)cc5)cccn4n3)c2)CC1]

or a pharmaceutically acceptable salt thereof.

The present disclosure also provides compositions for treating multiple sclerosis (MS) comprising Compound A, or a pharmaceutically acceptable salt thereof, and at least one pharmaceutically acceptable excipient.

The general description and the following detailed description are exemplary and explanatory only and are not restrictive of the disclosure, as defined in the appended claims. Other aspects of the present disclosure will be apparent to those skilled in the art in view of the detailed description of the disclosure as provided herein.

### BRIEF DESCRIPTION OF THE DRAWINGS

The summary, as well as the following detailed description, is further understood when read in conjunction with the appended drawings. For the purpose of illustrating the disclosure, there are shown in the drawings exemplary embodiments of the disclosure; however, the disclosure is not limited to the specific methods, compositions, and devices disclosed. In the drawings:

FIG. 1 depicts the effects of Compound A administration in vivo in an experimental allergic encephalomyelitis (EAE) mouse model of MS induced by rat Myelin Oligodendrocyte Lipoprotein Novatide amino acids 35-55 (MOG₃₅₋₅₅);

FIG. 2 depicts representative images and histological scoring for inflammation, demyelination, and axonal damage of lumbosacral sectioned spinal cords from an in vivo study of MOG-induced EAE in mice treated via the administration of Compound A;

FIG. 3 depicts effects of Compound A administration in vivo in an experimental allergic encephalomyelitis (EAE) mouse model of MS induced by mouse spinal cord homogenate (MSCH);

FIG. 4 depicts effects of Compound A administration in vivo in an experimental allergic encephalomyelitis (EAE) mouse model of MS induced by rat proteolipid protein amino acids 139-151 (PLP₁₃₉₋₁₅₁) in complete Freund's adjuvant (CFA) supplemented with Mycobacterium tuberculosis H37RA; and

FIG. 5 depicts pharmacokinetic data for administration of Compound A to canines.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the claimed disclosure.

As used in the specification including the appended claims, the singular forms “a,” “an,” and “the” include the plural, and reference to a particular numerical value includes at least that particular value, unless the context clearly dictates otherwise.

When a range of values is expressed, another embodiment includes from the one particular value and/or to the other particular value. All ranges are inclusive and combinable. Further, reference to values stated in ranges include each and every value within that range. When values are expressed as approximations, by use of the antecedent “about,” it will be understood that the particular value forms another embodiment. The term “about” as used herein when referring to a measurable value such as an amount, a temporal duration, and the like, is meant to encompass reasonable variations of the value, such as, for example, ±10% from the specified value. For example, the phrase “about 50%” can include ±10% of 50, or from 45% to 55%.

It is to be appreciated that certain features of the disclosure which are, for clarity, described herein in the context of separate embodiments, may also be provided in combination in a single embodiment. Conversely, various features of the disclosure that are, for brevity, described in the context of a single embodiment, may also be provided separately or in any subcombination.

### Terms

This disclosure relates to a 1,2,4-triazolo[1,5a]pyridine derivative, [8-(4-methanesulfonyl-phenyl)-[1,2,4]triazolo[1,5-a]pyridin-2-yl]-[3-(4-methyl-piperazin-1-yl)-phenyl]-amine, referred to as “Compound A” herein, having the following structure:

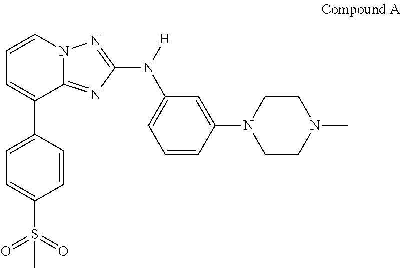 [SMILES: CN1CCN(c2cccc(Nc3nc4c(-c5ccc(S(C)(=O)=O)cc5)cccn4n3)c2)CC1]

or a pharmaceutical salt thereof, and its use in the treatment of multiple sclerosis. Compound A is a potent, orally active, small molecule inhibitor of JAK2. See, e.g., International Application No. PCT/US10/37363, U.S. Pat. Nos. 8,501,936 and 8,633,173, and U.S. Published Patent Application Nos. 2013/0267535 and 2014/0024655, each of which is incorporated by reference herein. Compound A can be prepared, for example, using methods analogous to Example 35 of International Application No. PCT/US10/37363.

As used herein, whether by itself or in conjunction with another term or terms, it should be understood that the phrases “method of treating” and “method of treatment” may be used interchangeably with the phrase “for use in the treatment of” a particular disease.

As used herein, whether by itself or in conjunction with another term or terms, “pharmaceutically acceptable” indicates that the designated entity such as, for example, e.g., a pharmaceutically acceptable excipient is generally chemically and/or physically compatible with other ingredients in a formulation or composition, and/or is generally physiologically compatible with the recipient thereof.

As used herein, whether by themselves or in conjunction with another term or terms, “subject(s),” “individual(s),” and “patient(s)”, refer to mammals, including humans. The term human(s) refers to and includes, a human child, adolescent, or adult.

As used herein, whether by themselves or in conjunction with another term or terms, “treats,” “treating,” “treated,” and “treatment,” refer to and include ameliorative, palliative, and/or curative uses and results, or any combination thereof. In other embodiments, the methods described herein can be used prophylactically. It should be understood that “prophylaxis” or a prophylactic use or result do not refer to nor require absolute or total prevention (i.e., a 100% preventative or protective use or result). As used herein, prophylaxis or a prophylactic use or result refer to uses and results in which administration of a compound or composition diminishes or reduces the severity of a particular condition, symptom, disorder, or disease described herein; diminishes or reduces the likelihood of experiencing a particular condition, symptom, disorder, or disease described herein; or delays the onset or relapse (reoccurrence) of a particular condition, symptom, disorder, or disease described herein; or any combination of the foregoing.

As used herein, whether used alone or in conjunction with another term or terms, “therapeutic” and “therapeutically effective amount”, refer to an amount of a compound or composition that (a) treats a particular condition, symptom, disorder, or disease described herein; (b) attenuates, ameliorates or eliminates one or more symptoms of a particular condition, disorder, or disease described herein; (c) delays the onset or relapse (reoccurrence) of a particular condition, symptom, disorder, or disease described herein. It should be understood that the terms “therapeutic” and “therapeutically effective” encompass any one of the aforementioned effects (a)-(c), either alone or in combination with any of the others (a)-(c).

As used herein, whether used alone or in conjunction with another term or terms, “therapeutic agent” refers to any substance included in a composition that is useful in the treatment of a disease, condition, or disorder or comorbidity (i.e., a disease, condition or disorder that exists simultaneously with MS) and is not Compound A.

Compound A and the pharmaceutically acceptable salts thereof may be administered alone, or in combination with one or more additional therapeutic agents as defined herein, in a pharmaceutical composition. An additional therapeutic agent may be used to treat one or more core symptoms and/or comorbidities associated with autoimmune disease in general or MS in particular. In one aspect, Compound A is formulated (and administered) with at least one therapeutic agent as a fixed dose. In another aspect, Compound A is formulated (and administered) separately from the therapeutic agent(s).

Some examples of therapeutic agents that may be used in combination with Compound A include, but are not limited to, e.g., alemtuzumab (Lemtrada®), alprostadil (MUSE®, Prostin VR®), amantadine (Lysovir®, Symmetrel®), amitriptyline (Elavil®, Triptafen®), azathioprine (Imuran®), baclofen (Lioresal®, Gablofen®), beta interferon 1a (Avonex®, Rebif®), beta interferon 1b (Betaferon®, Extavia®, Betaseron®), betamethasone, bisacodyl (Dulcolax®), botulinum toxin (Botox®), bupropion (Wellbutrin®), carbamazepine (Tegretol®), ciprofloxacin (Cipro®), clonazepam (Klonopin®, Rivotril®, Syn-Clonazepam®), clonazepam (Rivotril®), cyclophosphamide (Endoxana®), dalfampridine (Ampyra®), dantrolene (Dantrium®), darifenacin (Enablex®), desmopressin (Desmospray®, Desmotabs®, DDAVP nasal spray), dexamethasone, dextromethorphan+quinidine (Nuedexta®), diazepam (Valium®), dimethyl fumarate (Tecfidera®), docusate, duloxetine hydrochloride (Cymbalta®), fampridine (Fampyra®), fingolimod (Gilenya®), fluoxetine (Prozac®), gabapentin (Neurontin®), glatiramer acetate (Copaxone®), glycerin, hydroxyzine (Atarax®), ibuprofen, imipramine (Tofranil®), intravenous immunoglobulin (IVIg), isoniazid (Laniazid®, Nydrazid®), lamotrigine (Lamictal®), magnesium hydroxide (Phillips Milk of Magnesia®), meclizine (Antivert®), methenamine (Hiprex®), methotrexate (Maxtrex®), methylprednisolone (Solu-Medrol®), mineral oil, mitoxantrone (Novantrone®), modafinil (Provigil®), natalizumab (Tysabri®), nitrofurantoin (Macrodantim®), nortriptyline (Pamelor®, Aventyl®), oxcarbazepine (Trileptal®), oxybutynin (Ditropan®, Ditropan XL®, Lyrinel®, Oxytrol®), papaverine, paroxetine (Paxil®), peginterferon beta-1a (Plegridy®), phenazopyridine (Pyridium®), phenytoin (Dilantin®, Epanutim®), prazosin (Minipress®), prednisolone, prednisone (Deltasone®), pregabalin (Lyrica®), propanthetine (Pro-Banthine ®), psyllium hydrophilic musilloid (Metamucil®), sertraline (Zoloft®), sildenafil (Viagra®), sodium phosphate, sodium biphosphate, solifenacin succinate (Vesicare®), sulfamethoxazole (Bactrim®, Septra®), tadatafil (Cialis®), tamsulosin (Flomax®), terazosin (Hytrin®), teriflunornide (Aubagio®), tizanidine (Zanaflex®), tolterodine (Detrol®, Detrusitol®), trospium chloride (Sanctura®), vardenafil (Levitra®), and velafaxine (Effexor®).

The pharmaceutically acceptable salts of the compounds described herein include pharmaceutically acceptable acid addition salts, metal salts, ammonium salts, organic amine addition salts, and amino acid addition salts. Examples of acid addition salts include inorganic acid addition salts such as, for example, chloride (HCI), sulfate and phosphate salts, and organic acid addition salts such as, for example, acetate, maleate, fumarate, tartrate, citrate and lactate salts. Examples of metal salts include alkali metal salts such as, for example, lithium, sodium and potassium salts, and alkaline earth metal salts such as, for example, magnesium, calcium, aluminum, and zinc salts. Examples of ammonium salts include salts such as, for example, ammonium and tetramethylammonium salts. Examples of organic amine addition salts include salts such as, for example, morpholine and piperidine salts. Examples of amino acid addition salts include salts such as, fir example, glycine, phenylalanine, glutamic acid and lysine

Compound A can be formulated into a pharmaceutical composition (or simply “composition(s)” or “formulation(s)”) by admixture with one or more pharmaceutically acceptable excipients. As used herein, the terms “excipient” and “excipients” refer to and include any ingredient, other than Compound A and any other therapeutic agents, as defined herein, which may be present in a composition. Accordingly, pharmaceutically acceptable excipient(s) refer to and include ingredients such as, for example, surfactants, wetting agents, flavorings/taste masking agents, vehicles, carriers, diluents, preservatives, bulking agents, solubilizing agents, and the like. The choice of excipient(s) will largely depend on factors such as, for example, the particular mode of administration, as well as the desired solubility and stability profiles, as well as the nature of the dosage form.

Compositions comprising Compound A can be prepared for any number of different modes of administration, such as, for example, parenterally, particularly in the form of liquid solutions or suspensions; orally, particularly in the form of tablets, capsules or syrups/liquids; intranasally, particularly in the form of powders, nasal drops, or aerosols; or dermally, particularly in the form of gels, creams, lotions, or trans-dermal patches. Compositions comprising Compound A can be conveniently administered in unit dosage form and may be prepared by any of the methods well known in the pharmaceutical art, for example, as described in Remington's Pharmaceutical Sciences (Mack Pub. Co., Easton, Pa., 1980).

Tablets can be prepared using excipients such as lactose, glucose, sucrose, mannitol and methyl cellulose, disintegrating agents such as starch, sodium alginate, calcium carboxymethyl cellulose and crystalline cellulose, lubricants such as magnesium stearate and talc, binders such as gelatin, polyvinyl alcohol, polyvinyl pyrrolidone, hydroxypropyl cellulose and methyl cellulose, surfactants such as sucrose fatty acid ester and sorbitol fatty acid ester, and the like in a conventional manner. It is preferred that each tablet contains between about 0.01 mg to about 1500 mg of Compound A.

Granules can be prepared using excipients such as lactose and sucrose, disintegrating agents such as starch, binders such as gelatin, and the like in a conventional manner. Powders can be prepared using excipients such as lactose and mannitol, and the like in a conventional manner. Capsules can be prepared using gelatin, water, sucrose, gum arabic, sorbitol, glycerin, crystalline cellulose, magnesium stearate, talc, and the like in a conventional manner. The capsules may contain solid particles such as beads or, alternatively, be liquid or gel filled. It is preferred that each capsule contains between about 0.01 mg to about 1500 mg of Compound A.

Syrup preparations comprising Compound A can be prepared using sugars such as sucrose, water, ethanol, and the like in a conventional manner.

Ointments comprising Compound A can be prepared using ointment bases such as vaseline, liquid paraffin, lanolin and macrogol, emulsifiers such as sodium lauryl lactate, benzalkonium chloride, sorbitan mono-fatty acid ester, sodium carboxymethyl cellulose and gum arabic, and the like in a conventional manner,

Injectable preparations comprising Compound A can be prepared using solvents such as water, physiological saline, vegetable oils (e.g., olive oil and peanut oil), ethyl oleate and propylene glycol, solubilizing agents such as sodium benzoate, sodium salicylate and urethane, isotonicity agents such as sodium chloride and glucose, preservatives such as phenol, cresol, p-hydroxybenzoic ester and chlorobutanol, antioxidants such as ascorbic acid and sodium pyrosulfite, and the like in a conventional manner.

Formulations for parenteral administration may also contain polyalkylene glycols such as polyethylene glycol, hydrogenated naphthalenes and the like. In particular, biocompatible, biodegradable lactide polymer, lactide/glycolide copolymer, or polyoxyethylene-polyoxypropylene copolymers may be useful excipients to control the release of the active Compound A. Other potentially useful parenteral delivery systems for Compound A include ethylene-vinyl acetate copolymer particles, osmotic pumps, implantable infusion systems, and liposomes. Other formulations for parenteral administration may also include glycocholate for buccal administration, a salicylate for rectal administration, or citric acid for vaginal administration. Formulations for inhalation administration may contain excipients such as, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for trans-dermal patches are preferably lipophilic emulsions.

Compound A and the pharmaceutically acceptable salts thereof can be administered orally or non-orally, e.g., as an ointment or an injection. The concentrations of Compound A in a particular pharmaceutical composition can vary as described herein or as determined by one of skill in the art. In particular the concentration of Compound. A in a particular dosage form will depend upon factors such as the total dosage to be administered, the chemical characteristics (e.g., hydrophobicity) of Compound A, the route of administration, the age, body weight and symptoms of a patient, etc. The preferred dosage range of Compound A is likely to depend on variables such as the type and extent of progression of the disease to be treated, the overall health status of the particular patient, the particular formulation (dosage form)) and the excipients contained therein, as well as the route of administration.

The amount of Compound A, and pharmaceutically acceptable salts, can be administered in a single dose, once per day. Alternatively, the amount of Compound A can be administered two times per day. In other embodiments, the amount of Compound A can be administered three times per day. In still other embodiments, the amount of Compound A can be administered four times per day.

The skilled artisan will appreciate, based upon the description and examples provided herein, that the dosage range and dosing regimen for Compound A may be adjusted in accordance with methods well-known in the therapeutic arts. That is, one of skill in the art can readily determine the particular dose of Compound A and temporal requirements of administration needed to provide a detectable therapeutic benefit. Accordingly, it should be understood that while this application may describe and/or exemplify certain dose and administration regimens, these examples in no way limit the dose or administration regimens that may be used in practicing the methods described herein.

In one aspect, this disclosure describes and provides methods for treating multiple sclerosis comprising identifying an individual having multiple sclerosis or an individual susceptible to the development of multiple sclerosis and administering Compound A or a pharmaceutically acceptable salt thereof, to said individual

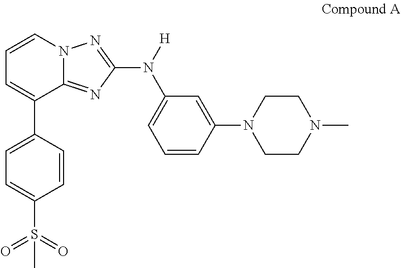 [SMILES: CN1CCN(c2cccc(Nc3nc4c(-c5ccc(S(C)(=O)=O)cc5)cccn4n3)c2)CC1]

In another aspect, this disclosure describes and provides methods according to the one above, wherein Compound A is administered up to four times per day. In another aspect, this disclosure describes and provides a method according to the first method in this paragraph, wherein Compound A is administered two times per day. In another aspect, this disclosure describes and provides a method according to the first method in this paragraph, wherein Compound A is administered one time per day.

According to the disclosure, Compound A, and the pharmaceutically acceptable salts thereof, can be administered in an amount of at least about 0.01 mg/kg per day. In some embodiments, Compound A is administered in an amount of about 0.01 mg/kg to about 1500 mg/kg per day. In other embodiments, Compound A is administered in an amount of about 3 mg/kg to about 300 mg/kg per day. In other embodiments, Compound A is administered in an amount of about 10 mg/kg to about 220 mg/kg per day. In other embodiments, Compound A is administered in an amount of about 60 mg/kg to about 220 mg/kg per day. In other embodiments, Compound A is administered in an amount of about 60 mg/kg to about 120 mg/kg per day. In another aspect, Compound A is administered in an amount of about 115 mg/kg per day. In other embodiments, Compound A is administered in an amount of about 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, or 1500 mg/kg per day.

In another aspect, this disclosure describes and provides a method according to any of those describe above, wherein Compound A is administered orally.

In another aspect, this disclosure describes and provides a method according to any of those describe above, further comprising administering a second therapeutic agent. In another aspect, this disclosure describes and provides a method according to the first method in this paragraph, wherein Compound A and the second therapeutic agent are administered separately. In another aspect, this disclosure describes and provides a method according to the first method in this paragraph, wherein Compound A and the second therapeutic agent are administered as a single dose.

In another aspect, this disclosure describes and provides compositions for use in treating an individual having multiple sclerosis or an individual susceptible to the development of multiple sclerosis comprising Compound A, or a pharmaceutically acceptable salt thereof:

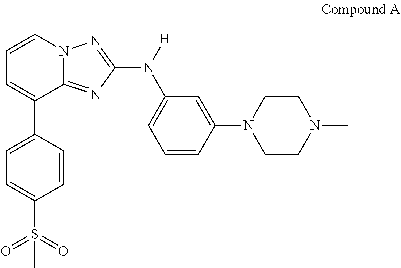 [SMILES: CN1CCN(c2cccc(Nc3nc4c(-c5ccc(S(C)(=O)=O)cc5)cccn4n3)c2)CC1]

and at least one pharmaceutically acceptable excipient. In another aspect, this disclosure describes and provides a composition according to the one above in a unit dose form. In another aspect, this disclosure describes and provides a composition according to the first composition of this paragraph in a tablet or capsule form.

According to the disclosure, Compound A, and the pharmaceutically acceptable salts thereof, can be present in compositions of the disclosure in an amount of at least about 0.01 mg/kg per day. In some embodiments, Compound A is present in an amount of about 0.01 mg/kg to about 1500 mg/kg per day. In other embodiments, Compound A is present in an amount of about 3 mg/kg to about 300 mg/kg per day. In other embodiments, Compound A is present in an amount of about 10 mg/kg to about 220 mg/kg per day. In other embodiments, Compound A is present in an amount of about 60 mg/kg to about 220 mg/kg per day. In other embodiments, Compound A is present in an amount of about 60 mg/kg to about 120 mg/kg per day. In another aspect, this disclosure describes and provides compositions wherein Compound A is present in an amount of about 115 mg/kg per day. In other embodiments, Compound A is present in an amount of about 5, 10, 20, 30, 40, 50, 60, 70, 80, 90, 100,110, 120, 130, 140, 150, 160, 170, 180, 190, 200, 210, 220, 230, 240, 250, 260, 270, 280, 290, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, or 1500 mg/kg per day.

Those skilled in the art will appreciate that numerous changes and modifications can be made to the preferred embodiments of the disclosure and that such changes and modifications can be made without departing from the spirit of the disclosure. It is, therefore, intended that the following examples and appended claims cover all such equivalent variations as fall within the true spirit and scope of the disclosure.

### EXAMPLES

Materials and Methods

Compound A was prepared in a manner analogous to the method described in Example 35 of International Application No. PCT/US10/37363.

Animal Models

Experimental allergic encephalomyelitis (EAE) is a Th1cell-mediated autoimmune disease model of multiple sclerosis. EAE shares many of the clinical and histopathological features of MS and is the accepted animal model for MS.

For the in-life portions of the following Examples, healthy, nulliparous, non-pregnant C57Bl/6 (for MOG-induced EAE studies), (Balb/c×SJL) F1 (CSFL) (for SCH-induced EAE studies) or SJL/J (for PLP-induced EAE studies) female mice were obtained from the Harlan Laboratories animal breeding center in Jerusalem, Israel. Animals weighed 17-20 grams upon arrival and were approximately 11 weeks of age. The body weights of the animals were recorded on the day of delivery. Healthy animals were assigned to study groups arbitrarily before treatment commenced. Mice were individually identified by markings on the body. Animal housing and care conditions were maintained according to the National Research Council (NRC) of Israel and the Teva Pharmaceutical Industries Ltd. Ethical Committee in an NRC-approved animal facility in Netanya, Israel.

For EAE induction in the following Examples, mice were injected subcutaneously (sc) in the flank with 200 μl of an encephalitogenic emulsion containing either 300 μg of rat Myelin Oligodendrocyte Lipoprotein Novatide amino acids 35-55 (MOG₃₅₋₅₅), 2 mg of a mouse spinal cord homogenate (MSCH), or 200 μg of a rat proteolipid protein amino acids 139-151 (PLP₁₃₉₋₁₅₁) in complete Freund's adjuvant (CFA) supplemented with 500 μg of Mycobacterium tuberculosis H37RA (MT) (Difco, Detroit, Mich.). For the MOG₃₅₋₅₅ injected and MSCH injected mice, pertussis toxin (PTX) was injected intraperitoneally (ip) on the day of induction and 48 hours later at a dose of 100 ng/0.2 ml/mouse. For the PLP injected mice, CFA (containing 1 mg/ml MT) was enriched with Mycobacterium tuberculosis to yield 2 mg/ml MT. For the MOG₃₅₋₅₅ injected mice, 30 mg of MOG was dissolved in 20 ml of normal saline to yield 1.5 mg/ml of MOG. The emulsions were made from equal parts of oil (20 ml CFS containing 2 mg/ml MT) and liquid portions (20 ml of 1.5 mg/ml MOG) in two syringes connected to each other with Leur lock to yield 0.75 mg/ml MOG. The dose of MOG in all the groups was 0.15 mg/0.2 ml/mouse, the dose of MT in all groups was 0.2 mg/0.2 ml/mouse. MOG-induced EAE is a chronic form of disease characterized by inflammation and demyelination. SCH-induced EAE is an acute and self-resolving form of the model which is primarily inflammation driven. PLP-induced EAE is a form of relapsing-remitting disease, used to model relapsing-remitting MS (RR-MS) in humans, with signs of inflammation, demyelination and axonal damage. A positive control reference agent was provided in each study to ensure consistent EAE responses across different models and study dates (data not shown herein).

Mice were randomly allocated to the following treatment groups: negative control (PBS), vehicle (0.5% methyl cellulose) and drug treated groups (Compound A). Fifteen mice from each group were used for EAE clinical observations. All cages were examined once daily for moribund individuals. The mice were monitored daily for clinical signs from the tenth day post-EAE induction daily until day 30 (MOG- and SCH-induced EAE) or day 60 (PLP-induced EAE). EAE clinical signs were defined as numerical scores as determined in a blinded fashion from a single person with more than 35 years of working with these models. The scoring method used herein was as follows: score of zero (0)=“Normal behavior, no neurological signs”; score of one (1)=“Tail weakness”; score of two (2)=“Hind legs weakness”; score of three (3)=“Hind legs paralysis”; score of four (4)=“Full paralysis”; score of five (5)=“Death”. For the PLP-induced EAE model, an additional parameter was inserted in between score 1-2, with a differentiation between a “limp tail or loss of righting reflex” and a “limp tail and loss of righting reflex”, this made moribund/death a score of six (6) in the PLP-induced model. All mice having scores of 1 and above were considered sick. Animals with a score of 5 for more than three days were given a score of 6 and sacrificed for humane reasons. For calculations purposes, the score of six for animals that were sacrificed or died was carried forward in the graphs.

Evaluation of EAE Clinical Signs, Calculations, and Scoring

Calculation of the incidence of disease (disease ratio): Incidence of disease equals the number of sick mice in treated group divided by the number of sick mice in control group. The percent inhibition according to incidence was calculated as one minus the number of sick mice in the treated group divided by the number of sick mice in the control group multiplied by 100%.

Calculation of the mortality/morbidity rate (mortality ratio): One minus the number of dead mice in treated group divided by the number of dead mice in control group multiplied by 100%.

Calculations of duration of disease: The total sum of disease duration of each mouse divided by the number of mice in the group. For calculation purposes, disease duration period for a mouse that did not develop EAE during the observation period was considered as 0 days.

Calculations of mean delay in onset of disease: The mean delay in onset of disease expressed in days was calculated by subtracting the mean onset of disease in control group from test group. For calculation purposes, the onset period for a mouse that did not develop EAE during the observation period was considered as 31 days.

Calculation of the mean maximal score and percent inhibition: The mean maximal score (MMS) of each group was calculated as the total sum of maximal score of each mouse divided by the number of mice in the group. Percent inhibition was calculated as one minus the MMS of treated group divided by the MMS of control group multiplied by 100%.

Calculation of the mean group score and percent inhibition: The daily scores of each mouse in the test group were summed and the individual mean daily score (IMS) was calculated as the total sum of daily score of the mouse divided by the observation period in days. The mean group score (GMS) was calculated as the total sum IMS of each mouse divided by the number of mice in the group. The percent inhibition was calculated by one minus the GMS of treated group divided by the GMS of control groups multiplied by 100%.

At the end of the observation period (day 30) the mice of the treatment groups were necropsied. The mice were deeply anesthetized with isoflourane and air and transcardially perfused, initially with a flushing solution (saline with heparin) until the perfusate was bloodless and then flushed with 4% buffered formalin. The vertebral columns (from the cervical region to the lumbar region) of each of the mice were dissected. The vertebral column was isolated by cutting away as much muscle as possible and sectioned distally at the last rib. The brain and the vertebral column were preserved in 4% buffered formalin.

Two transverse sections from each cervical and lumbar intumescence (cervico-thoracic and lumbo-sacral vertebral segments) were evaluated. Histological evaluation was performed on 4 μm thick sections stained with hematoxylin-eosin (H&E), Luxol fast blue periodic acid-Schiff (LFB/PAS) and Bielschowsky silver impregnation to assess inflammation, demyelination and axonal pathology. Histological sections were scored blind. Inflammation was evaluated for both gray and white matter, demyelination was evaluated within white matter only, and axonal injury was evaluated within the white matter. Scores from both cervico-thoracic, and lumbosacral sections, respectively, were averaged to obtain the average inflammation score and demyelination scores. The most severe area of demyelination for each section was used to evaluate axonal pathology. Semi-quantitative grading schematics were applied to evaluate inflammation, using hematoxylin-eosin stained-sections evaluated at 20× and 100× magnification (Inflammation severity score: 1=No inflammation; 2=perivascular/meningeal inflammation; 3=mild inflammation with the parenchyma; 4moderate inflammation within the parenchyma; 5=severe inflammation within the parenchyma) (Inflammation distribution score 1=None; 2=focal; 3=multifocal/patch; 4=regional; 5=multi-regional/confluent), and demyelination, using Luxol-fast blue/PAS stains-sections evaluated at 20× magnification (Demyelination severity score: 1=None; 2=minimal or rare spheroids/digestion chambers; 3=mild (few scattered spheroids/digestion chambers; 4=moderate; 5=severe) (Demyelination distribution score: 1=None; 2=focal; 3=multifocal/patchy; 4=regional; 5=multi-regional/confluent). For axonal pathology, the most severely affected region of one cervico-thoracic and one lumbo-sacral section from each animal was selected for evaluation by point sampling using a randomized 25-point Chalkey array eyepiece reticule superimposed on Bielschowsky's silver impregnation stained-sections at 400× magnification. The percentage axonal loss was determined by subtracting the number of points crossing axons subtracted by the total number of points of the stereological grid, divided by the total number of points of the stereological grid.

Significance for parameters measured over time was determined primarily by one-way ANOVA tests. For individual comparisons, areas under the curve (AUC) values were determined for each group with parameters measured over time. Mann-Whitney non-parametric and one- or two-way ANOVAs were used as statistical tests where noted in the descriptions of the Figures and Tables depending on the experiment and tested hypothesis. A p value <0.05 was considered significant. Statistical software used was GraphPad Prism® (vs. 5.01, 2007), and calculations were performed using the 2003 Office Professional version of Microsoft® Excel®.

### Example 1

### Treatment of MOG-Induced EAE

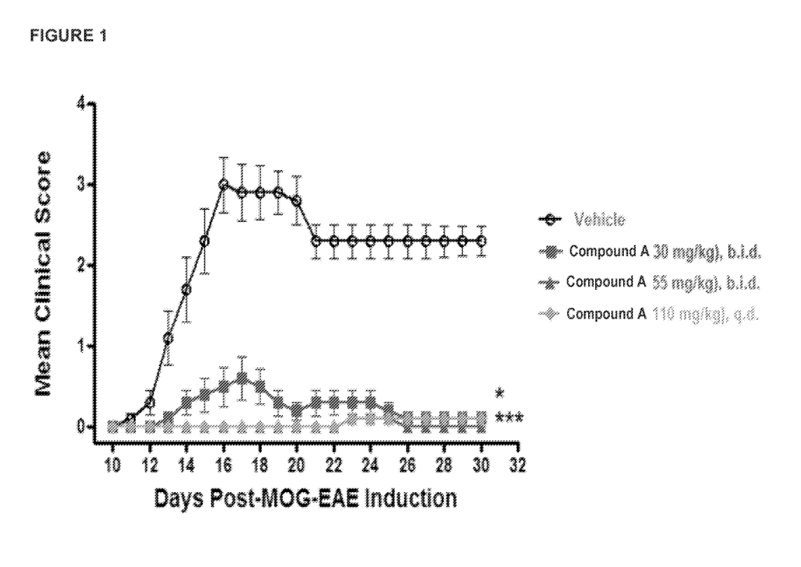

It was investigated if the treatment of animals with MOG-induced EAE with Compound A was possible and if any impact on the spinal cord inflammation and demyelination could be observed. Pharmacokinetics of Compound A dosed in vehicle in the C57Bl/6 laboratory mouse strain for doses 30-100 mg/kg were evaluated to ensure comparable exposure to previously tested models (data not shown). FIG. 1 shows the results of treatment of active MOG-induced EAE in mice with Compound A. Active EAE was induced on day 1 by the sc injection of encephalitogenic emulsion consisting of MOG₃₅₋₅₅ peptide and CFA at a volume of 0.2 ml/mouse in the right flank. PTX was injected intraperitoneally on the day of induction and 48 hours later at 100 ng/0.2 ml/mouse. Vehicle containing 0.5% methylcellulose or Compound A at 30, 55 or 110 mg/kg was administered orally (per os) either twice a day (all) or once a day (110 mg/kg only). Compound A was provided orally in suspension form in 0.5% methyl cellulose (Sigma, St. Louis, Mich.) to desired concentration for oral administration. All animals were monitored daily for clinical signs from the tenth day post-EAE induction until day 30. Scoring of EAE clinical signs was recorded on observations cards according to the grading system described herein. FIG. 1 shows Mean±SEM, with statistical calculations performed as 1-way ANOVA compared to Vehicle control group, N=15 mice per group, *p<0.05, ***p<0.001.

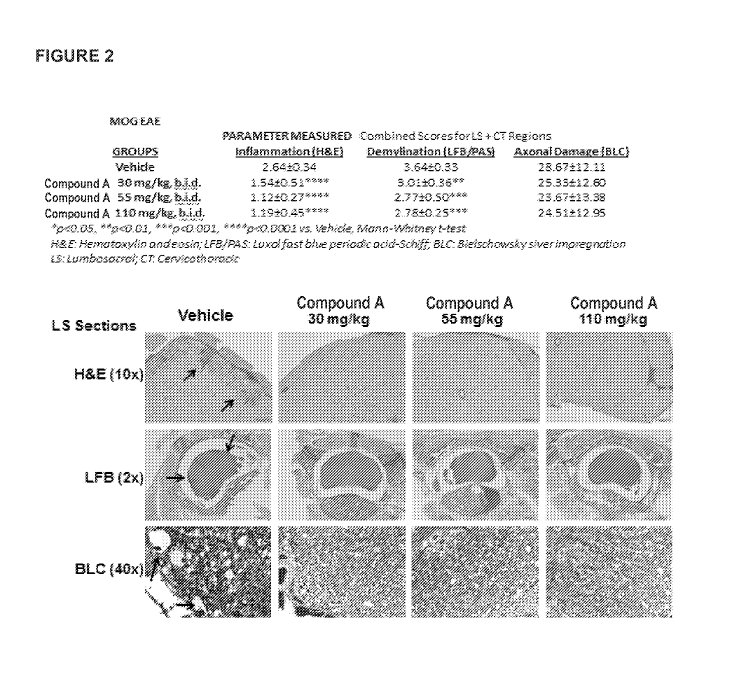

FIG. 2 shows values for histological section scoring for inflammation, demyelination, and axonal damage. Two transverse sections from each cervical and lumbar intumescence (cervico-thoracic and lumbo-sacral vertebral segments) were evaluated. Histological evaluation was performed on hematoxylin-eosin, Luxol fast blue periodic acid-Schiff (LFB/PAS) and Bielschowsky silver impregnation stained sections to assess inflammation, demyelination and axonal pathology respectively. Representative images from selected lumbosacral sectioned spinal cords are shown in the bottom portion of FIG. 2. Histological sections were scored blind by an independent board-certified pathologist. Inflammation was evaluated for both gray and white matter, demyelination was evaluated within white matter only, and axonal injury was evaluated within the white matter. Scores from both cervico-thoracic, and lumbosacral sections, respectively, were averaged to obtain the average inflammation score and demyelination scores. The most severe area of demyelination for each section was used to evaluate axonal pathology. Semi-quantitative grading schematics were applied to evaluate inflammation, using hematoxylin-eosin stained-sections evaluated at 20× and 100 × magnifications, and demyelination, using Luxol-fast blue/PAS stains-sections evaluated at 20× magnification. For axonal pathology, the most severely affected region of one cervico-thoracic and one lumbo-sacral section from each animal was selected for evaluation by point sampling using a randomized 25-point Chalkey array eyepiece reticule superimposed on Bielschowsky's silver impregnation stained-sections at 400× magnification. The percentage axonal loss was determined by subtracting the number of points crossing axons subtracted by the total number of points of the stereological grid, divided by the total number of points of the stereological grid.

Compound A treatment of MOG-induced EAE resulted in a significant decrease in clinical scores over the length of the study for each dose tested. Twice daily dosing of Compound A, orally, significantly reduced clinical scores, as compared to the vehicle control groups, 90.5% for 30 mg/kg (*p<0.05), 99.5% for 55 mg/kg (***p<0.001) and once daily dosing of 110 mg/kg, gave a 98.5% reduction in clinical score over that of vehicle (*p<0.001), as shown in FIG. 1. Mortality was not observed for any of the MOG-induced EAE treatment groups. Once daily dosing was also tested for Compound A at doses 10 mg/kg, 30 mg/kg and 60 mg/kg which no significant impact over vehicle was observed (data not shown).

TABLE 1
MOG-EAE Summary Table

| | | | Percent | | Percent | | Percent | | |
|---|---|---|---|---|---|---|---|---|---|
| Treatment | Mortality | Incidence | Inhibition-1 | MMS | Inhibition-2 | GMS | Inhibition-3 | Duration | Onset |
| Vehicle | 0/15 | 15/15 | — | 3.4 ± 0.8 | — | 2.1 ± 0.7 | — | 17.4 ± 1.7 | 11.5 ± 1.1 |
| Compound A | 0/15 | 7/15 | 53.3% | 0.7 ± 1.0 | 79.4% | 0.2 ± 0.4 | 90.5% | 3.7 ± 6.1 | 23.7 ± 8.2 |
| 30 mg/kg, | | | | | *** | | *** | | |
| twice a day | | | | | | | | | |
| Compound A | 0/15 | 1/15 | 93.3% | 0.1 ± 0.3 | 97.1% | 0.01 ± 0.04 | 99.5% | 0.2 ± 0.8 | 30.5 ± 2.1 |
| 55 mg/kg, | | | | | *** | | *** | | |
| twice a day | | | | | | | | | |
| Compound A | 0/15 | 1/15 | 93.3% | 0.1 ± 0.3 | 97.1% | 0.03 ± 0.1 | 98.5% | 0.5 ± 2.1 | 30.5 ± 2.1 |
| 110 mg/kg, | | | | | *** | | *** | | |
| twice a day | | | | | | | | | |

Table 1 shows results of the MOG-induced EAE study, showing mortality, incidence of disease, percent inhibition of first, second and third relapse and the group mean score (GMS) as calculated as the sum of individual mean daily score divided by the number of mice in the group. The statistical calculations performed were 1-way ANOVA compared to Vehicle control group, N=15 mice per group, *p<0.05, ***p<0.001.

A delay in the onset of clinical signs was observed in all groups treated with Compound A as shown in Table 1. This delay in disease onset correlated with in-life results, dose and frequency. Animals treated with Compound A at 55 and 110 mg/kg twice a day had an average disease onset of 30.5±2.1 days compared to 11.5±1.1 days in the vehicle groups. For comparisons, the onset of disease in 30 mg/kg twice a day treated groups was 23.7±8.2 days. The duration of disease in animals treated with Compound A at 30, 55 and 110 mg/kg, twice a day was 3.7±6.1, 0.2±0.8 and 0.5±2.1 days respectively, compared to 17.4±1.7 days in the vehicle control group. Thus, treatment with Compound A delayed disease onset and decreased disease duration in the MOG-induced model of EAE.

The mean maximal score (MMS) and group mean scores (GMS) both help determine the severity of the EAE clinical signs, onset and duration of disease, as seen in Table 1. The MMS and GMS of animals treated with 30 mg/kg, twice a day was 0.7±1.0 days (79.4% reduction, p<0.001) and 0.2±0.4 days (90.5% reduction, p<0.001) respectively, for 55 mg/kg, twice a day was 0.1±0.3 (97.1% reduction, p<0.001) and 0.01±0.04 days (99.5% reduction, p<0.001) respectively, and for 110 mg/kg, once a day, were 0.1±0.3 days (97.1% reduction, p<0.001) and 0.03±0.1 days (98.5% reduction, p<0.001) respectively, as compared to 3.4±0.8 and 2.1±0.7 days in the control vehicle group (Table 1). Histopathology was scored by an external blinded pathologist; scores are shown in FIG. 2. Histology samples were cut and stained according to the methods described herein. Groups treated with 30 mg/kg and 55 mg/kg twice a day, and 110 mg/kg once a day, Compound A, resulted in significantly reduced spinal inflammation (41.6%-57.5% reduction over vehicle, ****p<0.00001) and demyelination (17.3%-23.9% reduction over vehicle, **p<0.01) as determined using Hematoxylin and eosin and Luxol fast blue periodic acid-Schiff specialty stains.

### Example 2

### Treatment of SCH-Induced EAE

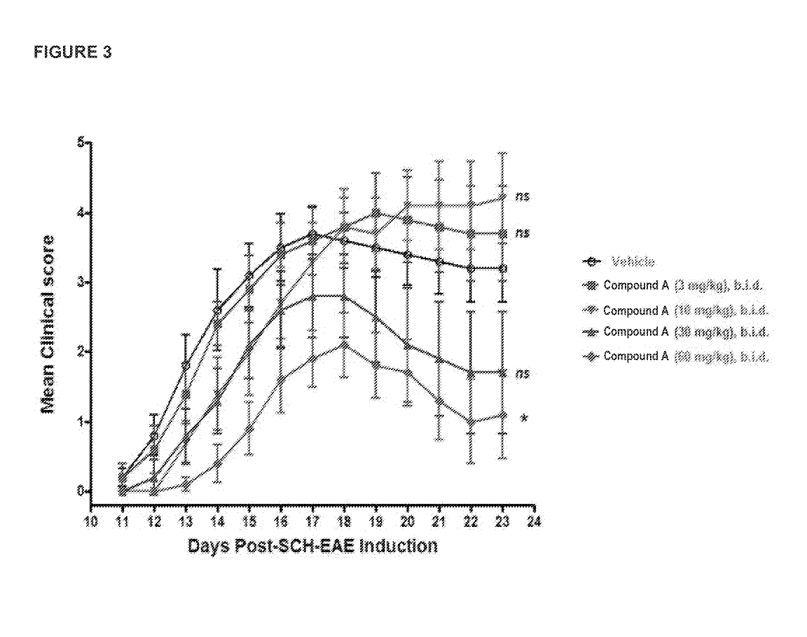

Studies were pursued in the acute mouse spinal cord homogenate induced model of EAE. FIG. 3 shows the results of treatment of active SCH-induced EAE in mice with Compound A. Active EAE was induced on day 1 by the sc injection of encephalitogenic emulsion consisting of mouse spinal cord homogenate (SCH) at 2 mg/mouse and CFA in a volume of 0.2 ml/mouse in the right flank. PTX was injected intraperitoneally on the day of induction and 48 hrs later at 125 ng/0.2 ml/mouse. Compound A was tested at doses 3, 10, 30, 60 mg/kg administered orally, twice a day and 10, 30, 60 mg/kg once a day (data not shown) in the SCH-induced EAE model, with comparison to vehicle containing 0.5% methylcellulose administered orally, twice a day. Compound A was provided orally in suspension form in 0.5% methyl cellulose (Sigma, St. Louis, Mich.) to desired concentration for oral administration. All animals were monitored daily for clinical signs from the tenth day post-EAE induction until day 30. Scoring of EAE clinical signs was recorded on observations cards according to the grading system described herein. FIG. 3 depicts a plot of Mean±SEM values; statistical calculations were performed as 1-way ANOVA compared to Vehicle control group, N=10 mice per group, “*” denotes p<0.05, “ns” denotes that results were not significant.

Compound A at 30 mg/kg, twice a day, and 60 mg/kg, twice a day, reduced the mean clinical scores, as compared to vehicle, as seen in FIG. 3, with “*” denoting p<0.05. Clinical score reductions for 60 and 30 mg/kg were 58.3% and 37.3% respectively. Mortality was more commonly observed in the SCH-induced EAE model than in the MOG-induced EAE model used in Example 1. 40% mortality was observed in the vehicle treated group and between 20-80% mortality was observed in groups treated with Compound A. The delay in onset of clinical signs was greatest for the 30 and 60 mg/kg twice a day groups with the 60 mg/kg twice a day group providing a significant effect, p<0.05, over vehicle control. The onset of disease in the 30 mg/kg and 60 mg/kg twice a day was 16.2±2.0 and 16.4±3.0 days respectively, compared to 13.1±2.0 days in the vehicle group. The duration of disease in groups treated with Compound A was also reduced for both the 30 and 60 mg/kg twice a day groups 8.2±3.3 and 8.4±4.2 days respectively, as compared to 10.9±2.0 days in the vehicle treated groups. The MMS and GMS of the 30 mg/kg twice a day treatment group was 3.3±1.3 days (17.5% reduction, p=0.25) and 1.73±1.4 days (37.3% reduction, p<0.05) respectively, compared to 4.0±.9 and 2.76±1.1 days in the vehicle control group. For the 60 mg/kg twice a day treated group, the MMS and GMS was 2.8±1.5 days (30% reduction, p=0.05) and 1.15±1.1 (58.3% reduction, p<0.01) days respectively, compared to 4.0±.9 and 2.76±1.1 days in the vehicle control group.

### Example 3

### Treatment of PLP-Induced EAE

Relapsing and remitting EAE as demonstrated by the PLP-peptide-induced model is characterized clinically by the development of ascending flaccid hind limb paralysis. The first clinical sign of diseases occurs in the acute phase in which mice show ascending paralysis following active disease induction. During the remission phase clinical improvement follows a clinical episode. During relapse increasing clinical disease is seen after remission and this is usually a one grade increase from the initial phase and is maintained for a couple days. Compound A was tested in a PLP-induced EAE model to determine if JAK2 inhibition could reduce the frequency and magnitude of relapses overtime.

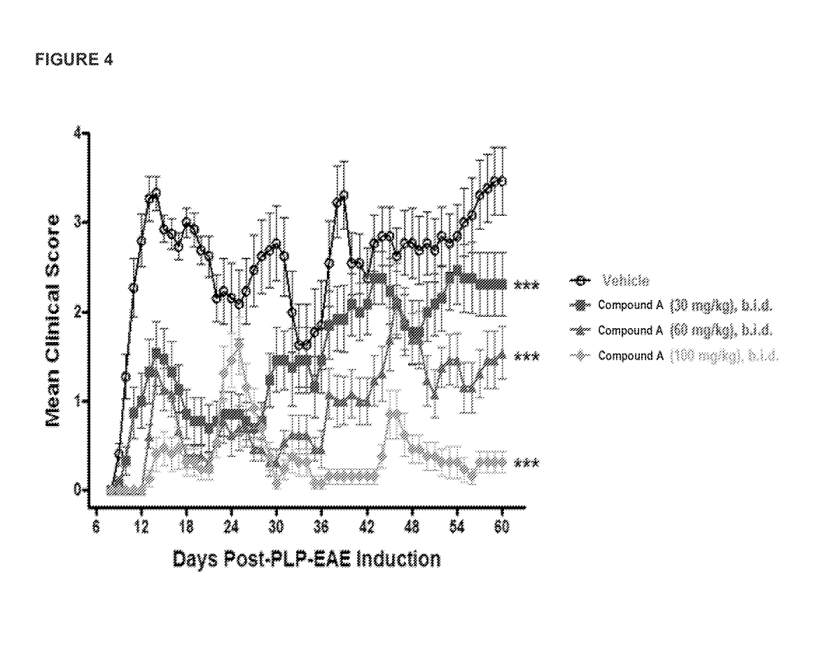

FIG. 4 shows the results of treatment of active PLP-induced EAE in mice with Compound A. Active EAE was induced on day 1 by the sc injection of encephalitogenic emulsion consisting of 1.5 mg/ml PLP₁₃₉₋₁₅₁ peptide and CFA at a volume of 0.2 ml/mouse in the right flank at 200 μl/mouse. PTX was injected intraperitoneally on the day of induction and 48 hours later at 100 ng/0.2 ml/mouse. Vehicle containing 0.5% methylcellulose or Compound A at 30, 60 or 100 mg/kg was provided orally, twice a day. Compound A was provided orally in suspension form in 0.5% methyl cellulose (Sigma, St. Louis, Mich.) to desired concentration for oral administration. All animals were monitored daily for clinical signs from the tenth day post-EAE induction until day 60. Scoring of EAE clinical signs was recorded on observations cards according to the grading system described herein. FIG. 4 shows Mean±SEM, with statistical calculations performed as 1-way ANOVA compared to Vehicle control group, N=16 mice per group, “***” denotes p<0.001.

TABLE 2

| | | | GMS | |
|---|---|---|---|---|
| | | | (1^{st} and 2^{nd} | Percent |
| Treatment | Mortality | Incidence | relapse) | Inhibition |
| Vehicle | 3/15 | 15/15 | 2.3 ± 0.7 | — |
| Compound A | 0/15 | 15/15 | 1.3 ± 0.9 | 43.5%*** |
| 30 mg/kg, | | | | |
| twice a day | | | | |
| Compound A | 0/15 | 15/15 | 1.4 ± 0.8 | 39.1%*** |
| 60 mg/kg, | | | | |
| once a day | | | | |
| Compound A | 0/15 | 15/15 | 0.8 ± 0.4 | 65.2%*** |
| 60 mg/kg, | | | | |
| twice a day | | | | |
| Compound A | 0/15 | 15/15 | 0.9 ± 0.5 | 60.9%*** |
| 100 mg/kg, | | | | |
| once a day | | | | |
| Compound A | 0/15 | 10/15 | 0.3 ± 0.3 | 87.0%*** |
| 100 mg/kg, | | | | |
| twice a day | | | | |

Table 2 shows results of the PLP-EAE study, showing mortality, incidence of disease, percent inhibition of first, second and third relapse and the group mean score (GMS) as calculated as the sum of individual mean daily score divided by the number of mice in the group. The statistical calculations performed were 1-way ANOVA compared to Vehicle control group, N=16 mice per group, “***” denotes p<0.001.

All doses provided significant responses in the PLP-EAE model, with the best overall response from the twice daily dosing regimen, as shown in FIG. 4 and Table 2. Dosing once daily with 60 and 100 mg/kg of Compound A provided highly significant reductions in clinical score over time as determined by 1-way ANOVA analysis (p<0.0001) with a 20.1% and 54.9% reduction in clinical score over that of vehicle (data not shown). The twice daily dosing of 30, 60 and 100 mg/kg Compound A provided 25.9%, 49.9% and 86.1% reductions in overall all mean clinical scores as compared to vehicle treated groups, as seen in FIG. 4, with “***” denoting p<0.001. There was a strong dose response with Compound A for both the once-a-day and twice-a-day dosing, as seen in FIG. 4.

Among the Compound A treated groups, the dose level of 100 mg/kg twice a day exhibited 97.6% activity (p<0.001) in the first relapse, 85.9% activity (p<0.001) in the second relapse, leading to the cumulative score, and 87% activity (p<0.001), according to the GMS of the first and second relapse calculated together (Table 2). PLP-induced EAE for first and second relapse scores combined for 100 mg/kg twice a day, 100 mg/kg once a day, 60 mg/kg twice a day, 60 mg/kg once a day, and 30 mg/kg twice a day Compound A dosing gave reductions of 87%, 60.9%, 65.2%, 39.1%, 43.5% respectively compared to vehicle controls, as shown in Table 2, with “***” indicating p<0.01. In ex vivo blinded pathology scoring, the 100 mg/kg dose provided significance in the reduction of inflammation (H&E staining, p<0.05, data not shown).

### Example 4

### Pharmacokinetics of Compound A in Canines

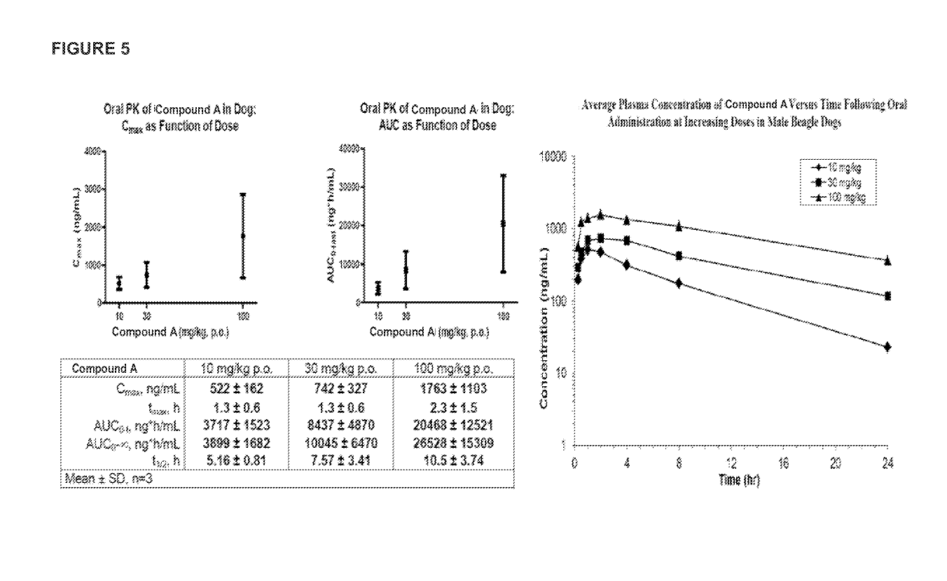

FIG. 5 shows pharmacokinetic data for administration of Compound A to canines 10, 30, and 100 mg/kg doses of Compound A were administered orally to male beagle dogs. Compound A was provided orally in suspension form in 0.5% methyl cellulose (Sigma, St. Louis, Mich.) to desired concentration for oral administration. Plasma concentration (ng/mL) of Compound A was measured at 15 minutes, 30 minutes, 1 hour, 2 hours, 4 hours, 8 hours, and 24 hours following oral administration, with average values (n=3) shown in FIG. 5. Ten and 30 mg/kg data was consistent with linear, dose-related increases in both the Cₘₐₓ and AUC. Emesis was observed in 2 out of the 3 dogs at 10 minutes after the 100 mg/kg dose; Cₘₐₓ levels at that dose may have been negatively impacted by the emesis. Cₘₐₓ for the 100 mg/kg dose was 1763 ng/ml, which is below the 2000-4000 ng/ml exposures that were associated with efficacy in mice.

The disclosures of each patent, patent application, and publication cited or described in this document are hereby incorporated herein by reference, in its entirety.

1. A method for treating multiple sclerosis in an individual having multiple sclerosis or in an individual susceptible to the development of multiple sclerosis comprising administering to said individual Compound A: 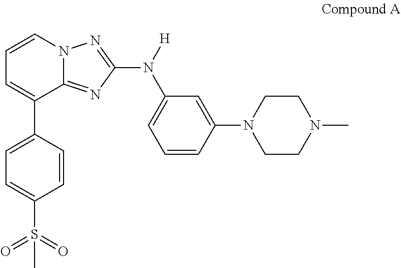 [SMILES: CN1CCN(c2cccc(Nc3nc4c(-c5ccc(S(C)(=O)=O)cc5)cccn4n3)c2)CC1]
or a pharmaceutically acceptable salt thereof.

2. The method of claim 1, wherein Compound A is administered up to four times per day.

3. The method of claim 1, wherein Compound A is administered two times per day.

4. The method of claim 1, wherein Compound A is administered one time per day.

5. The method of claim 1, wherein Compound A is administered in an amount of about 0.01 mg/kg per day to about 1500 mg/kg per day.

6. The method of claim 1, wherein Compound A is administered in an amount of about 3 mg/kg per day to about 300 mg/kg per day.

7. The method of claim 1, wherein Compound A is administered in an amount of about 10 mg/kg per day to about 220 mg/kg per day.

8. The method of claim 1, wherein Compound A is administered in an amount of about 60 mg/kg per day to about 220 mg/kg per day.

9. The method of claim 1, wherein Compound A is administered in an amount of about 60 mg/kg per day to about 120 mg/kg per day.

10. The method of claim 1, wherein Compound A is administered in an amount of about 115 mg/kg per day.

11. The method of claim 1, wherein Compound A is administered orally.

12. The method of claim 1, further comprising administering a second therapeutic agent.

13. The method of claim 12, wherein Compound A and the second therapeutic agent are administered separately.

14. The method of claim 12, wherein Compound A and the second therapeutic agent are administered as a single dose.

15. A composition for use in treating an individual having multiple sclerosis or an individual susceptible to the development of multiple sclerosis comprising
Compound A: 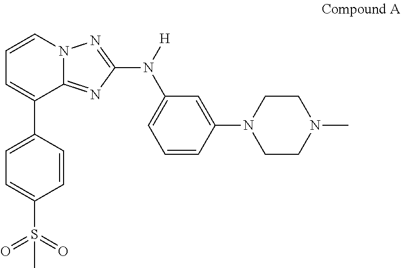 [SMILES: CN1CCN(c2cccc(Nc3nc4c(-c5ccc(S(C)(=O)=O)cc5)cccn4n3)c2)CC1]
or a pharmaceutically acceptable salt thereof, and
at least one pharmaceutically acceptable excipient.

16. The composition of claim 15, in a unit dose form.

17. The composition of claim 15, in a tablet or capsule form.

18. The composition of claim 15, wherein Compound A is present in an amount of about 0.01 mg/kg to about 1500 mg/kg.

19. The composition of claim 15, wherein Compound A is present in an amount of about 30 mg/kg to about 110 mg/kg.
